(19) United States
(12) Patent Application Publication
(10) Pub. No.: US 2014/0228805 A1
(43) Pub. Date: Aug. 14, 2014

(54) MODULAR INJECTION SYSTEM AND METHOD FOR DILUTING AN INJECTABLE FLUID

(71) Applicant: Allergan, Inc., Irvine, CA (US)

(72) Inventors: Christopher S. Mudd, Fort Worth, TX (US); Mitchell H. Babkes, Santa Clarita, CA (US); Zachary P. Dominguez, Santa Barbara, CA (US); Michael J. Prichard, Carlisle, MA (US); Martha E. Llewellyn, Maynard, MA (US); Shaohui Qiu, Belmont, MA (US); Lee F. Powers, Somerville, MA (US); Brett R. Johnson, Roseville, MN (US); Ahmet Tezel, Fort Worth, TX (US); Corey Josenhans, Santa Barbara, CA (US)

(73) Assignee: Allergan, Inc., Irvine, CA (US)

(21) Appl. No.: 14/253,303

(22) Filed: Apr. 15, 2014

Related U.S. Application Data
(63) Continuation of application No. 13/658,382, filed on Oct. 23, 2012.
(60) Provisional application No. 61/561,752, filed on Nov. 18, 2011.

(51) Int. Cl. A61M 5/14; A61M 5/172; A61M 5/142

(52) U.S. Cl. CPC A61M 5/1408; A61M 5/14244; A61M 5/172
USPC 604/506; 604/82

(57) ABSTRACT

A modular injection system enables a user to control the dilution ratio of mixed injectable fluid. In one embodiment, modular injection system includes a handheld injection device operatively connected to a separate control device. In one embodiment, the control device includes a drive unit configured to apply extrusion forces to fluids. In another embodiment, the handheld injection device includes the drive unit. In one embodiment, the modular injection system produces the mixed injectable fluid based on a selected dilution ratio. In one embodiment, the modular injection system produces the mixed injectable fluid based on selected injection rates.

### RELATED APPLICATIONS

This application is a continuation of U.S. patent application Ser. No. 13/658,382, filed Oct. 23, 2012, which claims priority to and the benefit of U.S. Provisional Patent Application No. 61/561,752, filed Nov. 18, 2011, the entire disclosure of each of these applications being incorporated herein by this reference.

### BACKGROUND

A number of medical and cosmetic applications involve controlled injection of substances into the body.

A medical syringe is a simple piston pump consisting of a plunger that fits tightly in a cylindrical barrel. The plunger can be pulled and pushed along inside the barrel, allowing the syringe to take in and expel a fluid through an orifice at the distal open end of the barrel. The distal end of the syringe is typically fitted with a hypodermic needle to introduce the barrel's fluid into a patient. Surprisingly, other than the materials used to make a syringe, conventional disposable syringes are much the same as the very earliest syringe designs.

Classic syringe/needle systems are far from optimal for the administration of today's injectable aesthetic compositions. Hydrogel-based dermal fillers can be quite difficult to inject using the conventional syringe/needle system or conventional injection techniques. Many dermal fillers are by their nature highly viscous, thus requiring relatively high extrusion forces, especially when injected through preferred fine gauge needles. Moreover, these materials can be injected into the face to correct wrinkles, including fine wrinkles as well as other minor defects in skin, and therefore, must be sometimes injected in trace amounts, and always with very high precision. Interestingly, these dermal fillers are commonly introduced into skin using standard needle and syringe combinations.

Using a traditional syringe, physicians can be required to supply possibly significant force, which may reduce the practitioner's ability to control the syringe. Further, traditional syringes typically require the user's hand to be placed a significant distance from the site of the injection in order to operate the plunger, which may also lead to inaccuracy. Automated injection machines, which supply the force required to perform the injection using a motor, may reduce some of these problems. However, some motorized injection devices have the disadvantage that they may be heavy and bulky. This added bulk and weight may lead to a lack of control because of user fatigue, balancing issues, etc.

As an additional complexity, it can be desired to mix fluids prior to or during an injection based on any number of factors such as, for example, the size of a patient's wrinkle. To increase user control of injections and accuracy of mixing injectable fluids, it is desirable to provide users with new types of injection devices. Accordingly, a need exists for further development of injection devices.

### SUMMARY

In one embodiment, injection devices can include: (a) at least one processor; (b) at least one input device operatively coupled to the processor; (c) a first cartridge that defines a first chamber which is configured to contain a first injectable fluid (e.g., a dermal filler); (d) a second cartridge that defines a second chamber which is configured to contain a second injectable fluid (e.g., a phosphate buffered saline); (e) a drive unit operatively coupled to the processor; (f) a mixing unit configured to mix the first injectable fluid and the second injectable fluid; and (g) at least one memory device storing instructions. In operation, when the instructions are executed by the at least one processor, the injection device selects a dilution ratio of the first injectable liquid and the second injectable liquid. In one embodiment, the injection device selects the injection ratio based on a user's input. Using the selected dilution ratio, the injection device produces an injectable mixed fluid by diluting the first injectable liquid with the second injectable liquid. Thereafter, the injection device extrudes the injectable mixed fluid.

In one embodiment, a modular injection system includes a handheld injector device and a control device which is separate from the handheld injector device. In one embodiment, the handheld injector device communicates with the control device using a communication wire. In one embodiment, the handheld injector device wirelessly communicates with the control device. In one embodiment, the handheld injector device includes a drive unit and a mixing unit which is configured to mix injectable fluids. In one embodiment, the control device includes the drive unit. The control device can include: (a) a first cartridge defining a first chamber configured to contain the first injectable fluid; and (b) a second cartridge defining a second chamber configured to contain the second injectable fluid. In one embodiment, the modular injection system enables a user to select a dilution ratio of the first injectable liquid and the second injectable liquid. Based on the selected dilution ratio, in one embodiment, the modular injection system can produce an injectable mixed fluid by diluting the first injectable liquid with the second injectable liquid.

In one embodiment, the control device is configured to be secured to a wrist of a user of the modular injection system.

In one embodiment, the drive unit includes gear motors and racks operatively coupled to the gear motors. In this example, the racks are operatively engaged with plungers. In another example, the drive unit includes a pressure source and a pressure regulator.

In one embodiment, the modular injection device selects an injection rate for the mixed injectable fluid. In this example, the modular injection device extrudes the injectable mixed fluid based on the selected injection rate. In another example, the modular injection device selects a first injection rate for the first injectable fluid, and selects a second injection rate for the second injectable fluid.

In some examples, the modular injection device may be configured to display any of the injection rates. In some examples, the modular injection device displays information indicating a volume of fluid injected.

Additional features and advantages are described herein, and will be apparent from, the following Detailed Description and Figures.

### BRIEF DESCRIPTION OF THE DRAWINGS

FIG. 1A illustrates a perspective view of one embodiment of a modular injection system disclosed herein.

FIGS. 1B and 1C illustrate perspective views of one embodiment of a control device disclosed herein.

FIG. 1D illustrates a cross-sectional perspective view of the control device, illustrating the drive unit having dual gear motors.

FIG. 1E illustrates a perspective view of one embodiment of the modular injection system, illustrating the control device being strapped to a user's wrist.

FIG. 1F illustrates a perspective view of one embodiment of the handheld injection device, illustrating the handheld injection device having a first portion which holds a second portion.

FIG. 1G illustrates a perspective view of one embodiment of the handheld injection device, illustrating the handheld injection device being gripped by a user.

FIG. 1H illustrates a schematic diagram of the modular injection system having an electronic configuration, illustrating a processor, a memory device, input devices and output devices.

FIGS. 2A, 2B and 2C illustrate front views of one embodiment of displays of the modular injection system, illustrating the selection of the dilution ratio and the injection rate.

FIG. 3 illustrates a perspective view of one embodiment of a component, illustrating two cartridges combined into one component.

FIG. 4A illustrates a cross-sectional perspective view of one embodiment of a single cartridge, illustrating the single cartridge having two chambers.

FIG. 4B illustrates a schematic diagram of one embodiment of a single cartridge, illustrating a regulator being used to control the dilution ratio of a combination of fluids.

FIG. 5 illustrates a cross-sectional perspective view of one embodiment of the mixing unit, illustrating the mixing unit having a half circle mixing path.

FIG. 6 illustrates a perspective view of one embodiment of the mixing unit, illustrating the mixing unit having a spiral mixing path.

FIG. 7 illustrates a cross-sectional perspective view of one embodiment of the mixing unit, illustrating the mixing unit having a helical mixing path.

FIG. 8 illustrates a cross-sectional perspective view of one embodiment of the mixing unit, illustrating the mixing unit having corrugated sections.

FIG. 9 illustrates a schematic diagram of one embodiment of the drive unit, illustrating the drive unit having independent dual gear motors.

FIG. 10 illustrates a schematic diagram of one alternative embodiment of the drive unit, illustrating the drive unit having a single gear motor and a transmission.

FIGS. 11A and 11B illustrate schematic diagrams of alternative embodiments of the drive unit, illustrating the drive unit being a pressure driven system.

FIG. 12 illustrates a schematic diagram of one alternative embodiment of the drive unit, illustrating the drive unit being a hydraulically driven system.

FIG. 13 illustrates a schematic diagram of one alternative embodiment of the drive unit, illustrating the drive unit having a nitinol driven system.

FIG. 14 illustrates a schematic diagram of one alternative embodiment of the drive unit, illustrating the drive unit having a wire system.

FIG. 15 illustrates a schematic diagram of one alternative embodiment of the drive unit, illustrating the control device having a pressure vessel and the handheld injection device having the cartridges.

FIG. 16 illustrates a schematic diagram of one alternative embodiment of the drive unit, illustrating the fluid lines of the hydraulic system being located in a tether.

FIGS. 17A and 17B are perspective views of one embodiment of the modular injection system, illustrating the handheld injection device including the drive unit and the cartridges.

### DETAILED DESCRIPTION

Described herein, generally are modular injection systems including a handheld injection device and a control device. In some embodiments, the control device is separate from the handheld injector device. In other embodiments, the control device is substantially separate from the handheld injection device or partially integrated with the handheld injection device.

In one embodiment, the handheld injection device includes a grippable housing and a mixing unit configured to mix injectable fluids to produce an injectable mixed fluid. In one embodiment, the control device includes: (a) cartridges configured to contain the injectable fluids; and (b) an injection drive mechanism or a drive unit configured to cause: (i) the mixing unit to mix the injectable fluids to mix; and (ii) the handheld injection device to extrude the injectable mixed fluid.

In the general operation of one embodiment, before an injection occurs, the control device can enable a user to select a dilution ratio of a first injectable fluid (e.g., hyaluronic acid (“HA”)) and a second injectable fluid (e.g., phosphate buffered saline (“PBS”)). As the injectable fluids move from their respective chambers in the control device to the needle in the handheld injection device for extrusion, using the mixing unit, the handheld injection device can dilute the first injectable liquid with the second injectable liquid based on the selected dilution ratio. In one embodiment, the control device also enables the user to control the rate at which the mixed fluid extrudes from the handheld injection device.

As mentioned above, a number of medical and cosmetic procedures involve the controlled injection of liquids, gels, and other fluids. For instance, procedures involving the injection of botulinum toxin or the injection of dermal fillers, may require highly controlled injections. Using the modular injection systems and methods disclosed herein, users need not supply some or all the force required to extrude the mixed injectable fluid. The modular injection systems and methods described herein can provide highly controlled injections by having the modular injection system: (i) supply the force which extrudes the injectable fluid through the needle; and (ii) extrude the fluid at a user controlled rate and with a user controlled dilution ratio, leaving the user free to concentrate on the injection itself, e.g., positioning of the needle.

Advantageously, the handheld injector device of the present disclosure can be lightweight and easy to manipulate and grip. Many of the heavier components of the modular injection system can be housed in the control device that is separate from the handheld injection device. In one embodiment, the control device is mountable to a user's arm or wrist to allow the user to view the display device while operating the handheld injector device within the same field of vision.

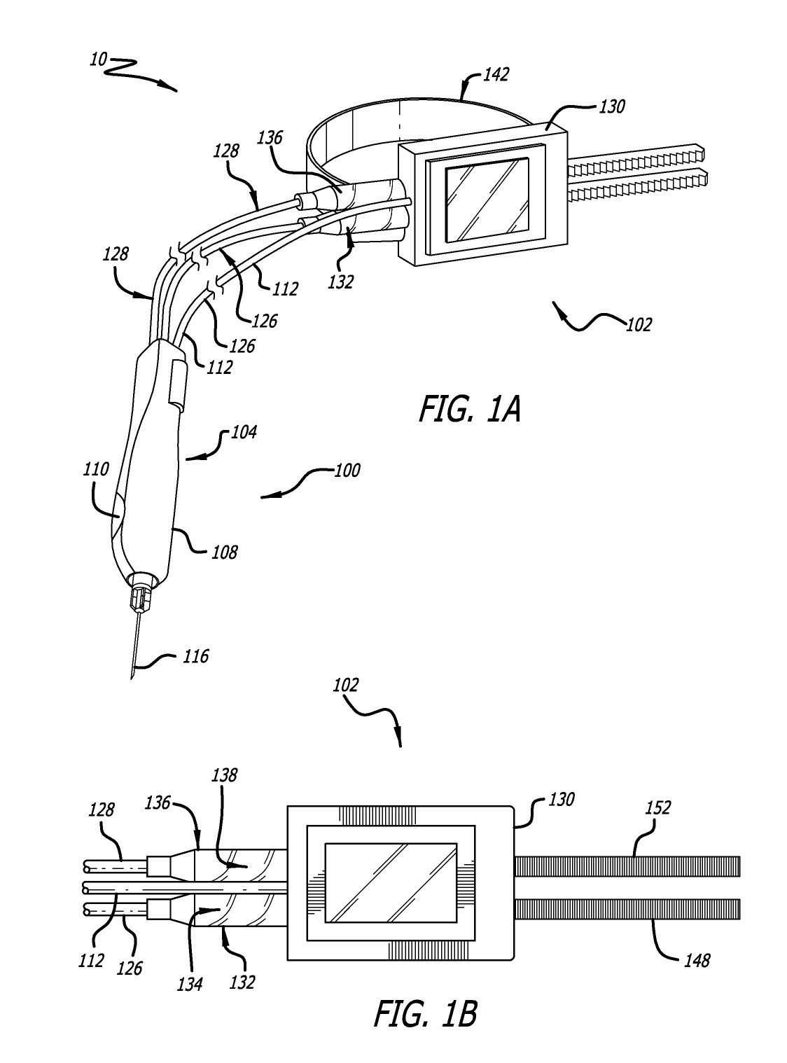

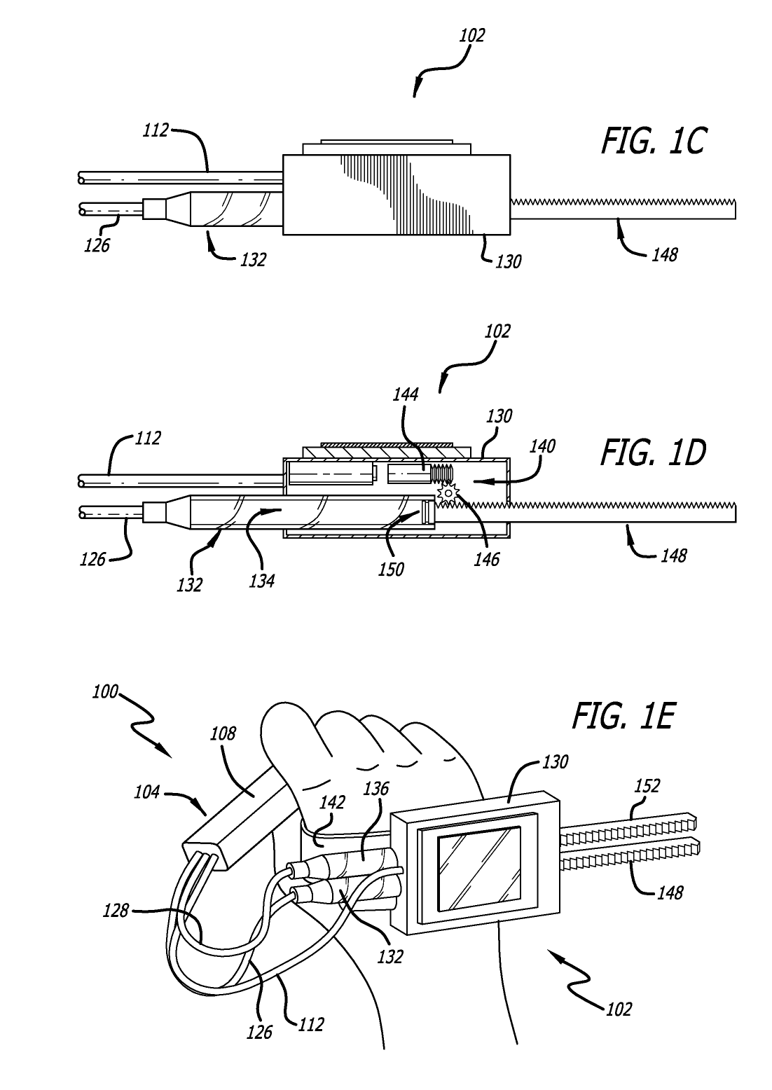

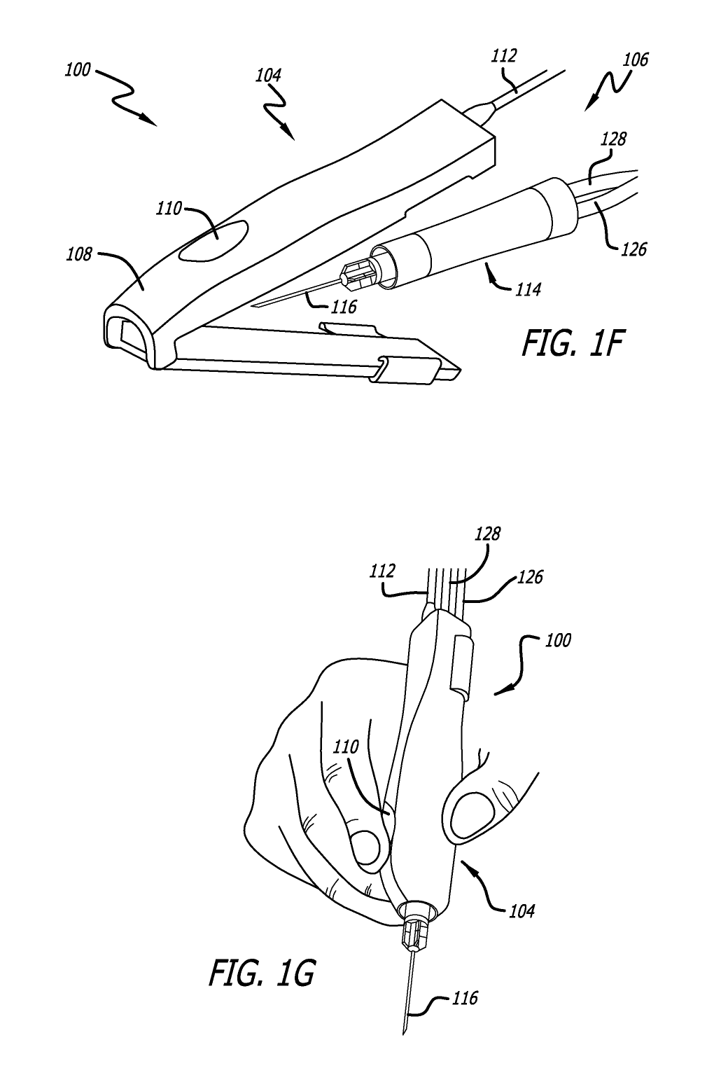

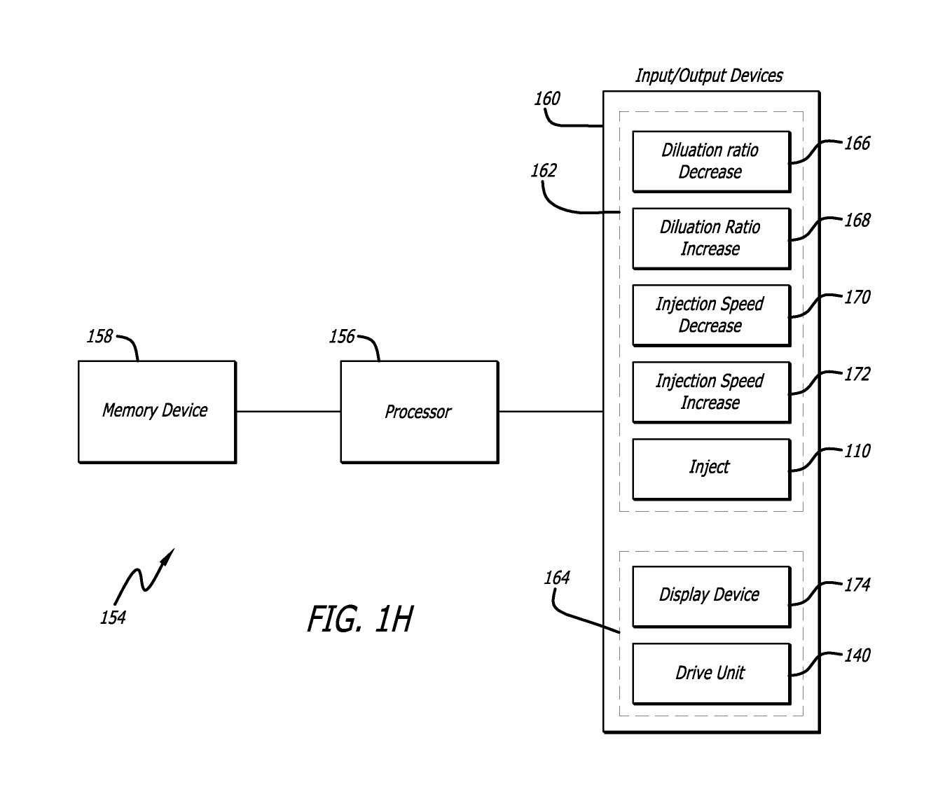

Referring now to FIGS. 1A to 1H, in one embodiment, modular injection system 10 includes handheld injector device 100 and control device 102 which is separate from handheld injector device 100.

In this embodiment, as illustrated in FIG. 1F, handheld injection device 100 includes first portion or handheld portion 104 and second portion or disposable portion 106. In this embodiment, second portion 106 is configured to slide into first portion 104. First portion 104 is configured to hold second portion 106. In one embodiment, after a desired amount of fluid has been injected into a patient, a user of the modular injection system may remove and discard the disposable portion 106.

In this embodiment, first portion 104 includes: (a) housing or body 108; and (b) input device or inject button 110. Using communication cable 112, inject button 110 is operatively connected to a processor located in control device 102 (discussed in more detail below). The inject button may start and stop the injection process.

As illustrated in FIG. 1F, in this embodiment, second portion 106 includes mixing unit 114 which is operatively connected to: (a) needle 116; (b) first tube 126; and (c) second tube 128. As illustrated in FIGS. 1A, 1B and 1E, first tube 126 and second tube 128 are also operatively connected to control device 102. In operation, drive unit 140 causes injectable fluids to flow from the control device to mixing unit 114 using first tube 126 and second tube 128. Mixing unit 114 is configured to mix injectable fluids.

Housing 108 may have a grippable housing, which may be made of any suitable material, e.g., metals, thermoplastics, thermoplastic elastomers (TPEs), silicones, glass, etc., or any combination of materials. Housing 108 may be shaped to comfortably accommodate a user's hand. A portion of housing 108 designed to be gripped may be textured to provide a secure grip, or may be covered in a layer of material designed to provide a secure grip.

In different embodiments, handheld injection device 100 may be ergonomically designed to facilitate injection for a wide variety of hand shapes, sizes and gripping positions. Advantageously, the handheld injector device can be easy to manipulate and grip.

In this embodiment, mixing unit 114 is configured to mix injectable fluids by directing the injectable fluids into half circle pathways. For embodiment, as illustrated in FIG. 5, mixing unit 114 defines: (a) first input channel 118; (b) second input channel 120; (c) half circle mixing path 122; and (d) output channel 124. In operation, as injectable fluids simultaneously move through half circle mixing path 122, half circle mixing path 122 diverts some injectable fluid and forces the diverted injectable fluid against the stream, encouraging turbulence.

In one embodiment, the mixing unit is configured to mix injectable fluids by directing the injectable fluids into a spiral mixing path. For example, as illustrated in FIG. 6, mixing unit 600 defines: (a) first input channel 602; (b) second input channel 602; (c) spiral mixing path 606; and (d) output channel 608. In operation, as injectable fluids simultaneously move through spiral mixing path 606, the injectable fluids can mix together to produce the injectable mixed fluid. In one embodiment, the mixing unit 600 is rotationally symmetric such that each piece can be mated to an identical piece.

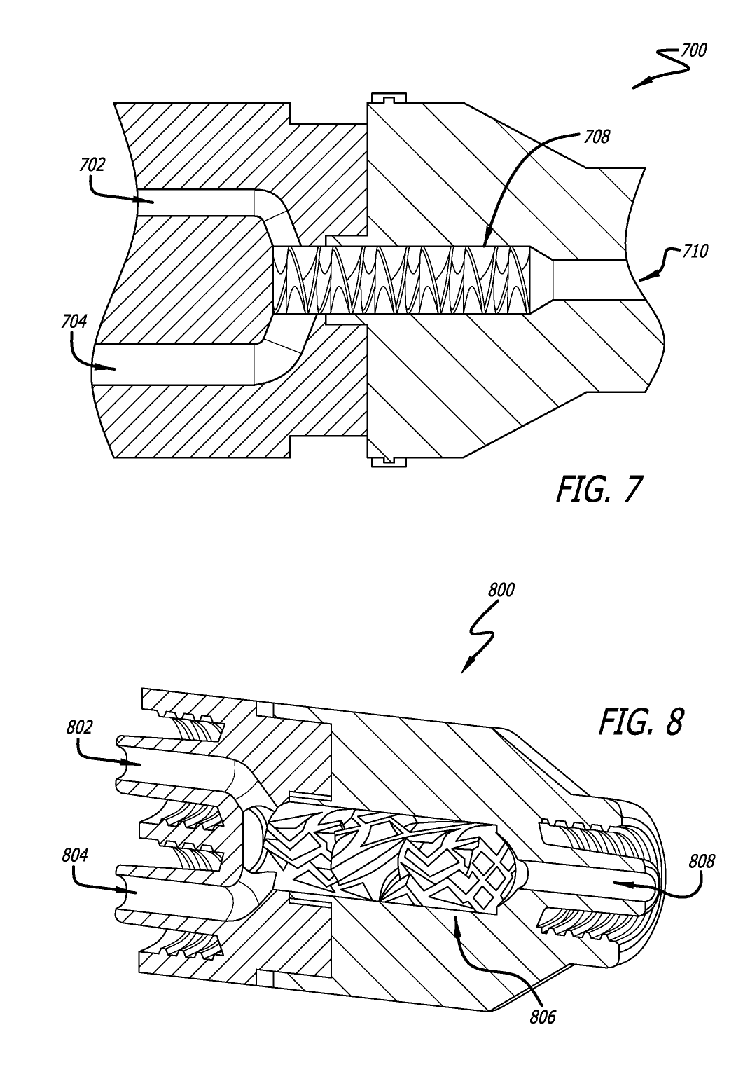

In one embodiment, the mixing unit is configured to mix the fluids by forcing the injectable fluids into a helical path. For example, as illustrated in FIG. 7, mixing unit 700 defines: (a) first input channel 702; (b) second input channel 704; (c) helical mixing path 708; and output channel 710. In operation, as injectable fluids simultaneously move through helical mixing path 708, the injectable fluids mix together to produce an injectable mixed fluid. Each mixer segment piece provides a single helical revolution in the opposite direction (i.e., clockwise vs. counterclockwise). The helical shape causes a significant amount of turbulence by causing the injectable fluids to change direction.

In one embodiment, the mixing unit includes corrugated sections which are configured to mix the injectable fluids. For example, as illustrated in FIG. 8, mixing unit 800 defines: (a) first input channel 802; (b) second input channel 804; (c) corrugated sections 806; and (d) output channel 808. In operation, as injectable fluids simultaneously move through the corrugated sections 806, the injectable fluids mix together to produce an injectable mixed fluid. In this embodiment, the corrugated sections are offset by 90° angles to each other. In each section, the corrugations run at 45° angles to the unobstructed injectable fluid path. The layers of corrugation with each section like 90° out of phase with each other.

Each of the mixing units described above are static. It should be appreciated that in other embodiments, the mixing unit may be dynamic. It should also be appreciated that in different embodiments, the injection device may include any suitable mixing unit, including any of the mixing units described herein.

As shown in FIGS. 1A and 1E, in this embodiment, handheld injection device 100 is attached to control device 102 using: (a) communication cable 112; (b) first tube 126; and (c) second tube 128. It should be appreciated that in different embodiments, where the modular injection system includes three or more cartridges containing injectable fluids, the handheld injection device may be connected to the control device using three or more tubes.

In one embodiment, handheld injection device 100 includes a power system. In this embodiment, handheld injection device 100 is connected to the control device using a cable or wire which supplies power to the power system from the control device. In one embodiment, where the handheld injector device houses certain components (e.g., a processor), the cable or wire is connected to these components. In one embodiment, handheld injector device 100 includes a motor, a processor and a power supply system configured to supply power to the motor and the processor. In this embodiment, power wires from the control device may be attached to the power system. In one embodiment, the cables or wires which connect the handheld injection device and the control device are removable.

In one embodiment, the handheld injector device wirelessly communicates with the control device. In one embodiment, the handheld injector device includes a transmitter and receiver, and the control device includes a transmitter and a receiver. In operation, using a wireless device, a communication channel may be established between the handheld injector device and the control device. Once established, the wireless communication channel may be used to exchange information and control signals between the handheld injector device and the control device as it would be exchanged using an embodiment with the cable. Because wireless communications have a greater chance of being disrupted than communications over a cable, the handheld injector device may be configured to react if wireless communication should be lost. For example, the handheld injector device may poll the control device periodically to sense whether it is in wireless communication with the control device. Should a poll fail, the handheld injector device may be configured to stop operation, continue operation using locally stored configuration parameters, and/or activate an alarm.

In one embodiment, the modular injection system includes both wireless and cable communication. For example, the handheld injector device and control device may each include wireless devices and a cable connection. In one embodiment, the wireless devices may not be used when the handheld injector device and the control device are connected via cable. In one embodiment, the handheld injector device houses an optional power source. For example, the handheld injector device may be configured to draw power over a cable, when attached the control device via cable. In this case, the optional power source need not be installed in the handheld injector device, reducing the weight of the handheld injector device. However, should the wireless devices be used for communication instead of the cable, the optional power source may be installed in the handheld injector device, which may be configured to draw power directly from the power source when in that configuration.

As illustrated in FIGS. 1A to 1E, in this embodiment, control device 102 includes: (a) housing or body 130; (b) first cartridge 132 defining first chamber 134 which is configured to contain the first injectable fluid; (c) second cartridge 136 defining second chamber 138 which is configured to contain the second injectable fluid; and (d) drive unit 140. It should be appreciated that, in different embodiments, the modular injection system may include three or more cartridges configured to contain injectable fluids.

In this embodiment, control device 102 is portable. In one embodiment, control device 102 may be strapped to the user's wrist via a VELCRO® strap or other typical strap or connector. For example, as illustrated in FIGS. 1A and 1E, control device 102 includes strap 142 which may be used to attach control device 102 to the user's wrist. In one embodiment, control device 102 can be attached to a chair, table or any surface. The strapping mechanism can be any mechanism known in the art, for example, a clasp, a buckle, a snap, a button, or the like. In one embodiment, control device 102 is not portable. For example, control device 102 may be a fixed unit into which the handheld injector device may plug.

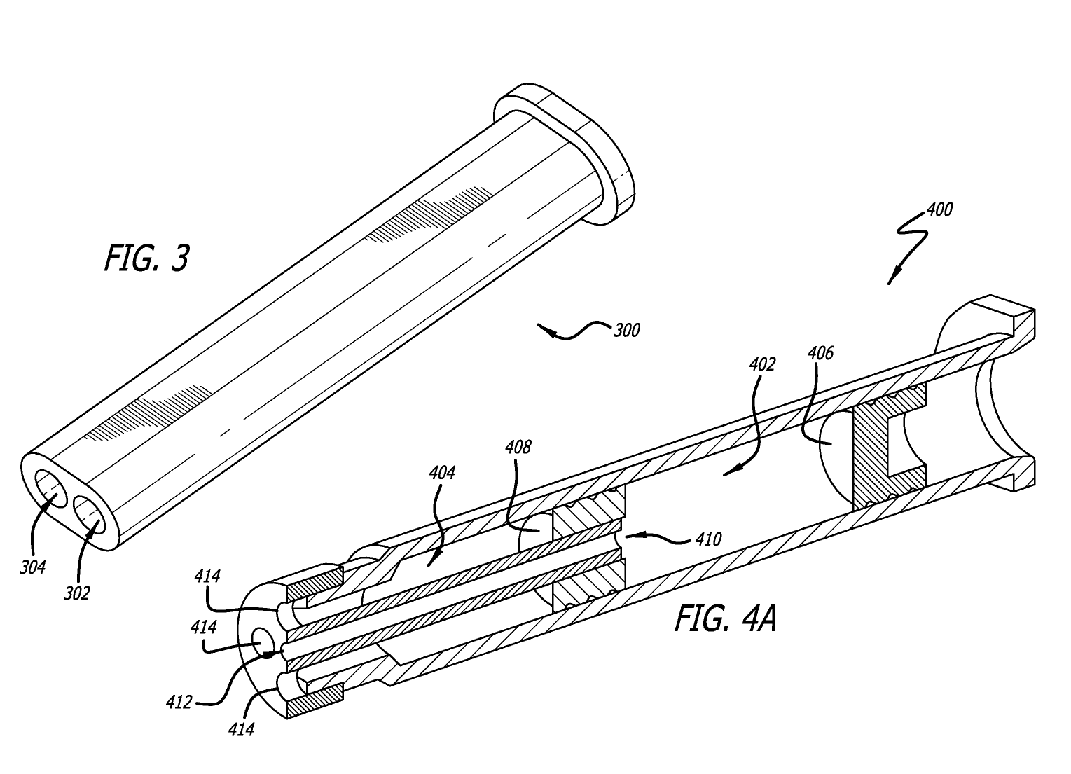

In this embodiment, first cartridge 132 is separate from second cartridge 136. In an alternative embodiment, cartridges may be combined into a single component. For example, as illustrated in the FIG. 3, cartridge 300 includes two cartridges combined into a single component. Cartridge 300 defines first chamber 302 for containing a first injectable fluid and second chamber 304 for containing a second injectable fluid. In this embodiment, chambers 302 and 304 are configured to receive different plungers to facilitate the extrusion process. The geometry of these cartridges are not fixed, as long as they can hold a minimum of 1 mL of fluid.

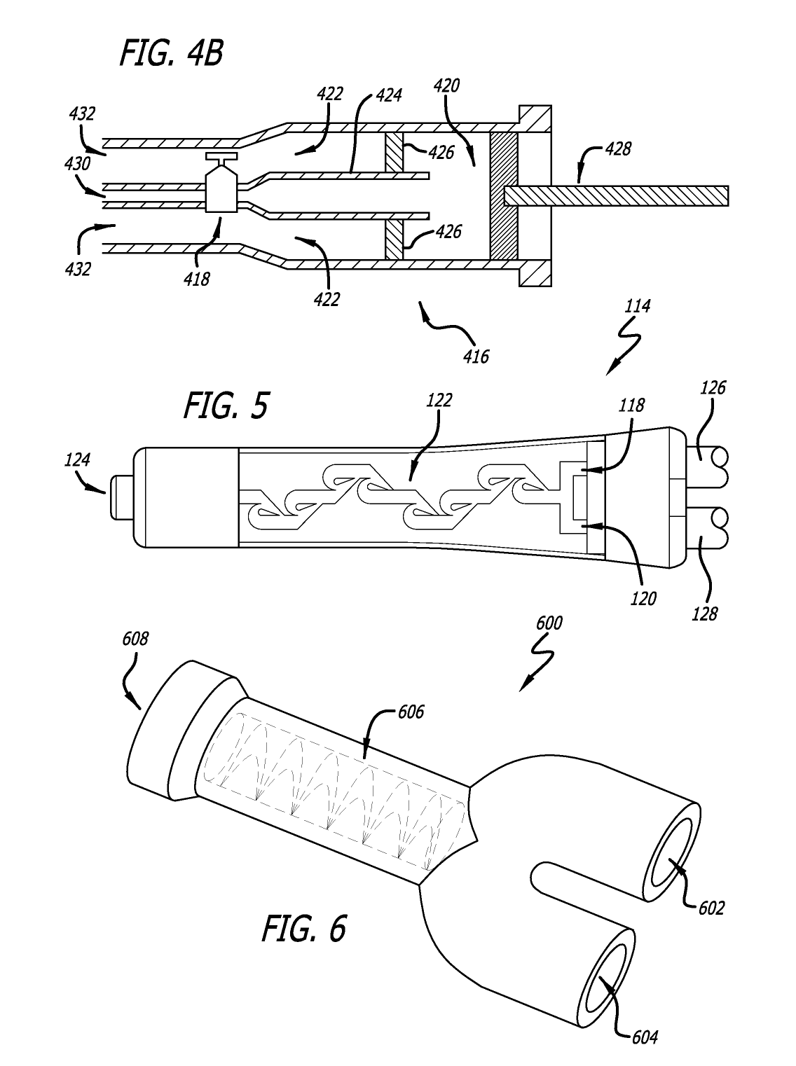

In one alternative embodiment, control device 102 includes a single cartridge which defines a plurality of chambers which contain the fluids. Referring to FIG. 4A, single cartridge 400 defines: (a) first chamber 402 configured to contain a first injectable fluid; and (b) second chamber 404 configured to contain a second injectable fluid. In this embodiment, single cartridge 400 includes first plunger head 406 and second plunger head 408. Single cartridge 400 forms center channel 410. In operation, when first plunger head 406 is pushed forward, the first injectable fluid is caused to flow from first chamber 402 through center channel 410 and out the end 412 of the center channel 410. In response to second plunger head 408 being pushed forward, the second injectable fluid is caused to flow from second chamber 404 through output channels 414 of single cartridge 400.

In one embodiment, where modular injector system 10 includes a single cartridge, the single cartridge is operatively connected to a flow/pressure regulator. For example, as illustrated in FIG. 4B, single cartridge 416 is operatively connected to flow/pressure regulator 418. Single cartridge 416 defines: (a) first chamber 422 configured to contain a first injectable fluid; and (b) second chamber 420 configured to contain a second injectable fluid. In this embodiment, single cartridge 416 has center stem 424. Single cartridge 416 includes first plunger 426 which has a hole portion and forms a seal around center stem 424. In operation, when second plunger 428 is pushed forward, the first injectable fluid is caused to flow through center stem 424 and out of the end 430 of center stem 424. In response to first plunger 426 being pushed forward, the second injectable fluid is caused to flow through output channel 432 of single cartridge 416. In this embodiment, when flow/pressure regulator 418 is in a closed position, only the second injectable fluid from the front half is allowed to flow out from single cartridge 416. As flow/pressure regulator 418 is opened, a greater percentage of the first injectable fluid from the back half can be driven out. In this embodiment, the dilution ratio of the mixed fluid is determined based on the amount of fluid flow through flow/pressure regulator 418. In this embodiment, using encoders (not shown), modular injection system 10 monitors the amount of fluid which has passed. Modular injection system 10 determines the location of first plunger 428 using any one of the described methods herein. Modular injection system 10 determines the location of second plunger 426 by monitoring the flow out of any one of channels 430 and 432, or by using a linear encoder (not shown) which determines the linear position of second plunger 426. In this embodiment, flow/pressure regulator 418 is positioned in line with center stem 424. In an alternate embodiment, flow/pressure regulator 418 is positioned in line with outer channel 432. It should be appreciated that the energy source which drives the plunger can be any suitable energy source such as any of the energy sources described herein (e.g., gear motors, pressure source, nitinol actuators, etc.).

In one embodiment, the modular injection system attaches to and operates a standard needle and cartridge combination. That is, in this embodiment, the modular injection system does not include any cartridges. Rather, the modular injection system is configured to receive the cartridges. In one embodiment, the modular injection system is attached to the cartridges using a luer slip or luer lock attachment. In one embodiment, the cartridges include a protruding or snap feature used to lock the cartridges into the modular injection system when it is fully inserted. In another embodiment, the inner body of the handheld injection device includes the protruding or snap feature. In other embodiments, the control device includes the protruding or snap feature. In one embodiment, the cartridge includes a ring which seals the cartridge into a cartridge slot of the modular injection system.

The modular injection system may also include a cartridge retention and ejection mechanism. This mechanism may facilitate loading of, e.g., pre-filled, disposable cartridges. The mechanism may also provide for the rotation of cartridges. In one embodiment, the handheld injection device includes the cartridge retention and ejection mechanism. In another embodiment, the control device includes the cartridge retention and ejection mechanism.

In one embodiment, the modular injection system houses chambers itself to contain fluids to be injected. The chambers can be housed in the handheld injection device. In this embodiment, a needle is attached directly to the handheld injection device. The chambers can also be housed in the control device. In one embodiment, the modular injection system includes a cartridge housing in which the cartridge(s) may be secured. In another embodiment, the cartridge housing is substantially in the form of a tube. The cartridge housing may be designed to hold a disposable, pre-filled cartridge. The cartridge housing may be all or partially transparent, allowing a user to view the cartridge during operation. For example, the cartridge housing may provide a user with a view of both a cartridge in the housing and also a cartridge plunger which may extrude fluid from the cartridge when the modular injection system is in operation.

In one embodiment, the cartridge(s) is made of cyclic olefin copolymer (COC). Any other suitable materials may be utilized.

In one embodiment, each cartridge is filled using different dedicated filling tips. Once filled, a sealing tip may be employed to prevent mixing of the injectable fluids while in storage.

In one embodiment, the cartridge includes a needle or is configured to be coupled to a needle. In one embodiment, using a luer tip, the at least one cartridge is coupled to a needle. The needle itself may have any suitable gauge, for example, a gauge between about 10 and about 33. In one embodiment, the needle is a 30 G×¾″ needle.

In one embodiment, after a desired amount of fluid has been injected into the patient, the user of the injection device may remove and discard the used cartridge(s) along with the needle.

It should be appreciated that, in different embodiments, modular injection system 10 is configured to include or attach to any suitable cartridge.

In one embodiment, the injectable fluids (e.g., the first injectable fluid and/or the second injectable fluid) include at least one biocompatible material. These biocompatible materials include, but are not limited to, dermal fillers, hyaluronic acid-based dermal fillers (e.g., Juvederm™ Ultra and Juvederm™ Ultra Plus (Allergan, Irvine, Calif.)), hydrogels (i.e., superabsorbent natural or synthetic polymers), organogels, xerogels, encapsulated and/or cross-linked biomaterials, silicones, glycosaminoglycans (e.g., chondroitin sulfate, dermatin sulfate, dermatin, dermatin sulfate, heparin sulfate, hyaluronic acid, o-sulfated hyaluronic acid), polysaccharides (e.g., chitosan, starch, glycogen, cellulose), collagen, elastin, local anesthetics (e.g., Benzocaine, Chloroprocaine, Cyclomethycaine, Dimethocaine/Larocaine, Propoxycaine, Procaine/Novocaine, Proparacaine, Tetracaine/Amethocaine, Amino amides, Articaine, Bupivacaine, Carticaine, Cinchocaine/Dibucaine, Etidocaine, Levobupivacaine, Lidocaine/Lignocaine, Mepivacaine, Piperocaine, Prilocaine, Ropivacaine, Trimecaine), drugs, bioactive agents, antioxidants, enzyme inhibitors (e.g., anti-hyaluronidase), vitamins, minerals, water, saline, light curable or light activated materials, vaccines, and pH curable or pH activated materials. Other biocompatible materials not mentioned above are also considered within the scope of the present description.

In one embodiment, the second injectable fluid includes a bioactive agent which facilities delivery of the first injectable fluid injection (e.g., to reduce extrusion force and/or viscosity). Additional bioactive agents may include anti-proliferatives including, but not limited to, macrolide antibiotics including FKBP-12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids. Drugs can also refer to bioactive agents including anti-proliferative compounds, cytostatic compounds, toxic compounds, anti-inflammatory compounds, anti-fungal agents, steroids, chemotherapeutic agents, analgesics, antibiotics, protease inhibitors, statins, nucleic acids, polypeptides, growth factors and delivery vectors including recombinant micro-organisms, liposomes, and the like. Combinations of additional bioactive agents are also within the scope of the present description.

Other injectable fluids (e.g., the first injectable fluid and/or the second injectable fluid) may include toxins such as botulinum toxins. The botulinum toxin can be selected from the group consisting of botulinum toxin types A, B, C₁, D, E, F and G, a pure or purified (i.e., about 150 kD) botulinum toxin, as well as a native or recombinant botulinum toxin. The material can comprise between about 1 unit to about 20,000 units of the botulinum toxin or a therapeutically effective amount, and the composition can comprise an amount of botulinum toxin sufficient to achieve a therapeutic effect lasting between 1 month and 5 years. The botulinum toxin can be reconstituted within the device as described elsewhere herein or before the cartridge is placed in the device. The botulinum toxin can be reconstituted with sterile 0.9% sodium chloride (saline).

In one embodiment, the dilution ratio is 1 to 100 units of botulinum toxin per 0.1 mL of saline. More preferably, in one embodiment, the dilution ratio is 1 to 50 units per 0.1 mL of saline, or 1 to 10 units per 0.1 mL of saline. In one embodiment, 4 units per 0.1 mL of saline are used. The dilution ratio can be highly dependent on the type of botulinum toxin used or combination of botulinum toxins used.

As illustrated in FIG. 1D, in this embodiment, drive unit 140 includes first gear motor 144 and second gear motor (not shown). First gear motor 144 is operatively connected to first gear 146, and the second gear motor is operatively connected to a second gear (not shown). In this embodiment, drive unit 140 also includes: (a) first rack 148 which is operatively engaged with the first gear 146 and first plunger 150; and (b) second rack 152 which is operatively engaged with the second gear and a second plunger. It should be appreciated that drive unit 140 illustrated in FIG. 1D may provide an effectively infinite number of dilution ratios and injection speeds by independently setting the speed of one gear motor relative to another gear motor.

As illustrated in FIGS. 1A, 1E and 1F, in this embodiment, first tube 126 has: (i) a first end operatively connected to first cartridge 132; and (ii) a second end operatively coupled to mixing unit 114. Second tube 128 has: (i) a first end operatively connected to second cartridge 136; and (ii) a second end operatively coupled to mixing unit 114.

In operation, in this embodiment, drive unit 140 drives linear motion of the plungers causing mixed injectable fluid to be extruded. More specifically, first gear motor 144 causes first gear 146 to turn, thereby driving the linear motion of first rack 148. First rack 148 engages first plunger 150, thereby causing the first injectable fluid to flow from the first chamber, through first tube 132, to mixing unit 114 of handheld injection device 100. The second gear motor causes the second gear to turn, thereby driving the linear motion of second rack 152. Second rack 152 engages the second plunger, thereby causing the second injectable fluid to flow from the second chamber, through second tube 136, to mixing unit 114 of handheld injection device 100.

In one embodiment, the rotational output of the motors drives the linear motion of the racks through worm gears. In another embodiment, the rotational output of the motors drive the linear motion of the racks through concentric gearing of an internally threaded gear to a threaded rack.

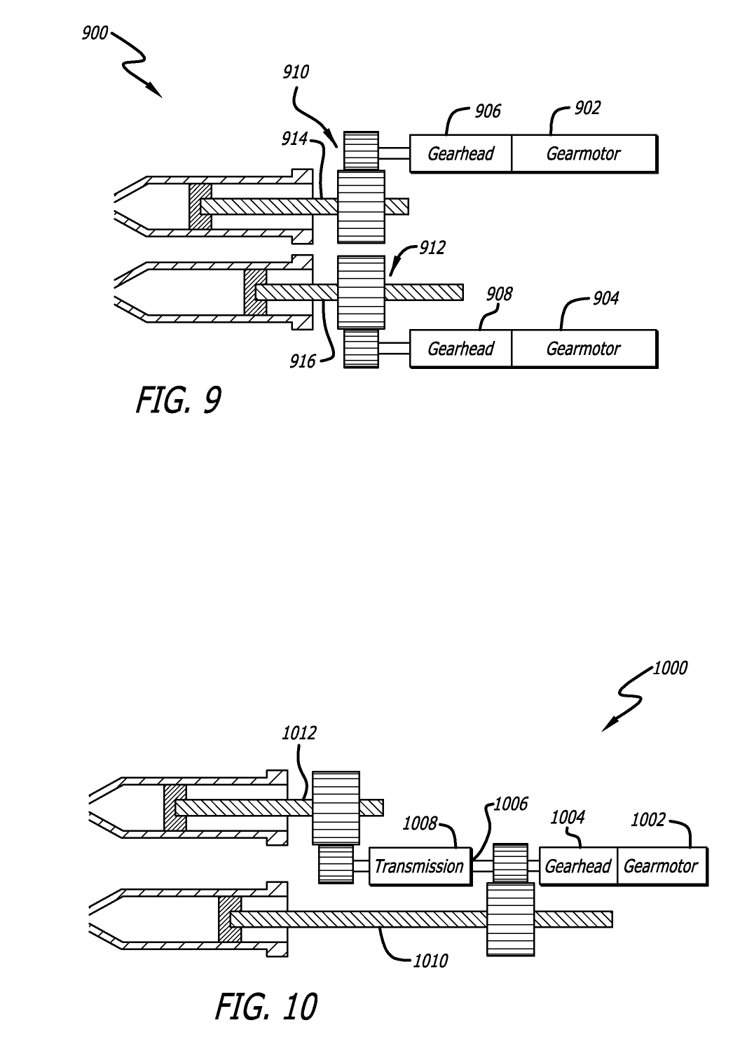

FIG. 9 illustrates a schematic diagram of one embodiment of the drive unit, illustrating the drive unit having independent dual gear motors. Drive unit 900 includes: (a) gear motors 902 and 904; (b) gearheads 906 and 908; (c) gear assemblies 910 and 912; and (d) plungers 914 and 916. In operation, gear motors 902 and 904 are driven through gearheads 906 and 908 to achieve necessary speed reduction and force multiplication. The rotation output from gear motor 902 drives the linear motion of plunger 914 through gear assembly 914. Similarly, the rotation output from gear motor 904 drives the linear motion of plunger 916 through gear assembly 912. Described in more detail below with reference to FIGS. 10 to 16, in different embodiments, the drive unit may include a single gear motor and a transmission, a pressure driven system, a hydraulically driven system, a nitinol driven system, or a wire system.

It should be appreciated that any of the motors discussed herein may be any suitable electric motor capable of supplying the necessary force. In one embodiment, the motors are operatively connected to the plungers via certain of the drive units discussed herein. In some embodiments, the drive units function to transfer the rotational motion of the motors into the linear motion of the plunger.

In one embodiment, modular injection systems can include a control system. The control system can include at least one processor, at least one memory device operatively connected to the at least one processor, at least one input device operatively connected to the at least one processor, and at least one output device operatively connected to the at least one processor. For example, as illustrated in FIG. 1H, the modular injection system of FIGS. 1A to 1H, includes control system 154 having: (i) processor 156; (ii) memory device 158; and (iii) input/output devices 160.

The at least one processor may be any suitable processor unit of a kind normally used in such devices. In one embodiment, the control system can include one or more digital processors, such as a digital microprocessor or a micro-controller based platform. In one embodiment, the control system includes one or more analog control units such as a suitable integrated circuit or one or more application-specific integrated circuits (ASICs). In one embodiment, the control system is in communication with, or operable to access or exchange signals with the at least one memory device. In this embodiment, the memory device stores program code or instructions, executable by the processor(s), to control the injection device. In one embodiment, such memory device can include: (a) RAM (MRAM); (b) ferroelectric RAM (FeRAM); (c) read only memory (ROM); (d) flash memory; (e) EEPROM (electrically erasable programmable read only memory); or a suitable combination of such memory devices. It should be appreciated that any other suitable magnetic, optical, or semiconductor memory may operate in conjunction with, or as part of, the injection device.

In one embodiment, the output devices include at least one display device. In one embodiment, the display device includes an LCD screen which is located on a front of the injection device, and allows a user to interact with the system. In one embodiment, the LCD screen displays a dot matrix pattern. In one embodiment, the display device includes LED technology. In one embodiment, the modular injection system causes an LED display device to display proprietary artwork. In one embodiment, the display device includes electroluminescent panels. In one embodiment, the display device includes an interface. Using the interface, the user may control the operation of the device.

The modular injection system may be configured to cause the display device to display at least one of, for each fluid contained: (i) the volume that has been injected; (ii) the volume remaining; (iii) the starting volume; and (iv) the speed or injection rate. The display device may also display at least one of: (a) the total volume of fluid that has been extruded or injected; (b) the speed or rate of injection of the mixed fluid; (c) the dilution ratio of the fluid being injected. In addition, other information may be displayed to facilitate different functions. For instance, the display device may also display configuration screens, summary information, error indicators in the case of a malfunction, and/or battery power information.

In one embodiment, the input devices include inject button 110. The inject button may be located on handheld injection device 100 in a position which is conveniently accessible by a user's fingers or thumb during injection. The inject button may start and stop the injection process. In one embodiment, the user may press and hold the inject button to begin the injection, and may release the inject button to stop the injection. In other embodiments, the injection process may work in other ways. For instance, the user may press the inject button once to begin the injection and a second time to stop the injection. In other embodiments, the injection process starts based on a user pressing switch or some other actuator.

In one embodiment, control system 154 includes at least one input device (e.g., a keypad, a button, a dial or a switch) which enables a user to control the overall speed or rate or volume of the extrusion. In one embodiment, control system 154 includes at least input device (e.g., a button, dial or switch) which enables a user to control the overall speed or rate or volume of the injection by enabling the user to independently control the speed or rate or volume of the injection of each injectable fluid.

In one embodiment, the modular injection system is configured to extrude fluid at a plurality of predetermined selectable speeds. As described in more detail below, in one embodiment, the modular injection system is configured to extrude fluid at the following four different selectable speeds: very low, low, medium and high. In one embodiment, the modular injection system is configured to extrude fluid at a dynamic speed which enables extrusion of each of the four different speeds based on the amount of pressure exerted on the inject button. Lighter pressure on the inject button corresponds to a lower injection speed and a higher pressure corresponds to a higher injection speed. Approximate corresponding flow rates are shown in Table 1.

These flow rates were determined based on evaluation physician's typical extrusion rates.

TABLE 1
Exemplary Injection Rates

| Speed Setting | Injection Rate (+/−0.20 mL/minute)* |
|---|---|
| Very Low | 0.30 |
| Low | 0.60 |
| Medium | 0.90 |
| High | 1.20 |
| Dynamic | 0.30-1.20 |

*APPROXIMATE INJECTION RATE

In one embodiment, the input devices include at least one encoder. Using at least one encoder, the modular injection system determines the position of the plungers. For example, the modular injection system illustrated in FIGS. 1A through 1H may use a first encoder (not shown) to determine the position of the first plunger, and use a second encoder (not shown) to determine the position of the second plunger. In this embodiment, these encoders can be located on the gear motors. In one embodiment, using the at least one encoder, the modular injection system determines and displays volume information of each contained fluid and/or the total volume extruded/injected.

In one embodiment, the encoder is a rotational encoder connected to a motor. In this embodiment, the rotational encoder can be configured to sense the rotation of the motor. For example, the motor may rotate a portion of the rotational encoder.

In different embodiments, other portions of the modular injection system may be encoded. For example, in one embodiment, the modular injection system includes a separate linear encoder for each of the plungers.

Referring to FIG. 1H, control system 154 includes at least one processor 156; at least one memory device 158 operatively connected to processor 156; input devices 162 operatively coupled to processor 156; and output devices 164 operatively coupled to processor 156. In this embodiment, as illustrated in FIGS. 1A, 1B and 1E, input devices 162 include: (a) dilution ratio decrease button 166; (b) dilution increase button 168; (c) injection speed decrease button 170; (d) injection speed increase button 172; and (e) inject button 110. Output devices 164 include: (a) display device 174; and (b) the drive unit 140. In this embodiment, handheld injection device 100 includes inject button 110 which is operatively coupled to processor 156. It should be understood that, in this embodiment, certain portions of the control system are included in the handheld injection device and other portions of the control system are included in the control device. In one embodiment, control system 115 is a portion of a control system for the entire modular injection system (not shown).

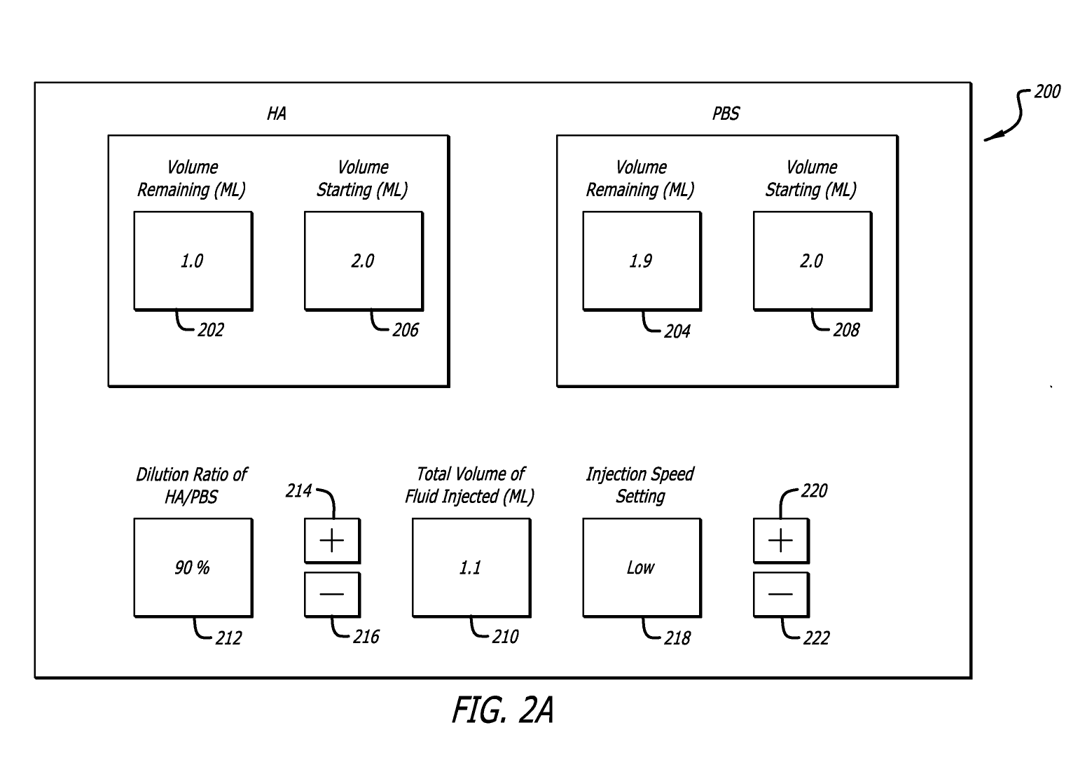

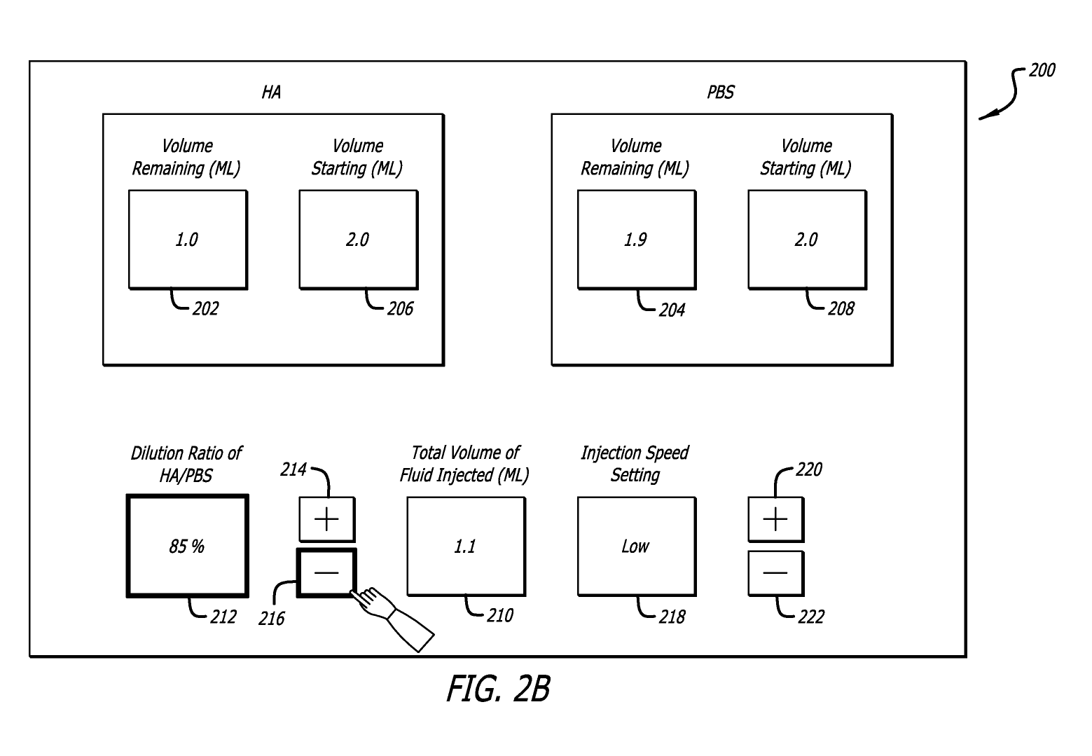

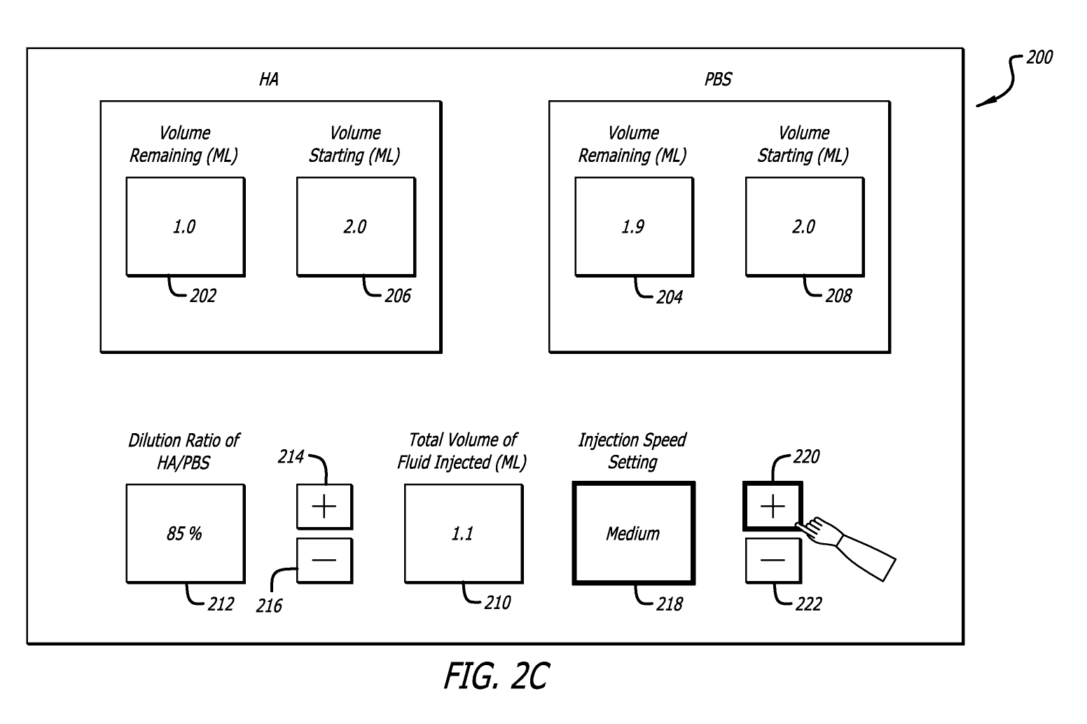

Referring to FIGS. 2A to 2C, this embodiment generally illustrates: (a) the selection of a dilution ratio of injectable fluids to produce a mixed fluid; and (b) for the mixed fluid, the selection of an injection or extrusion speed. In this embodiment, the first injectable fluid is HA and the second injectable fluid is phosphate buffered saline PBS. In this embodiment, the modular injection system provides a mixed fluid made up of HA and PBS based on the selected dilution ratio, and extrudes the injectable mixed fluid based on the selected injection speed. It should be understood that although in this embodiment, the fluids include HA and PBS, in different embodiments the fluids may include any suitable fluid which is desired to be diluted or mixed.

FIG. 2A illustrates a point in time in which 1.1 mL of injectable fluid had previously been extruded from the modular injection system.

In this embodiment, display device 200 displays first volume remaining meter 202 for the HA, and second volume remaining meter 204 for the PBS. First volume remaining meter 204 displays an amount or volume of HA remaining. At the point in time illustrated in FIG. 2A, first volume remaining meter 202 indicates that 1.3 mL of HA remain. Second volume remaining meter 204 displays an amount or volume of PBS remaining. At the point in time illustrated in FIG. 2A, second volume remaining meter 204 indicates that 1.9 mL of HA remain.

Display device 200 also displays first volume starting meter 206 for the HA, and second volume starting meter 208 for the PBS. First volume starting meter 206 displays an amount or volume of HA which the injection device started with before the extrusion process. In this embodiment, first volume starting meter 206 indicates that, before the extrusion process, the injection device included 2.0 mL of HA. Second volume starting meter 208 displays the amount or volume of PBS which the injection device started with before the extrusion process. In this embodiment, second volume starting meter 208 indicates that, before the extrusion process, the injection device included 2.0 mL of PBS.

Display device 200 can also displays total volume of fluid injected or extruded meter 210. Total volume of fluid injected meter 210 displays the total amount or volume of fluid which has been injected or extruded. At the points in time illustrated in FIGS. 2A to 2C, the total volume of fluid injected meter 210 indicates that 1.1 mL of total fluid had previously been injected.

Display device 200 also displays dilution ratio meter 212. In this embodiment, dilution meter 212 displays the ratio of HA to PBS. At the point in time illustrated in FIG. 2A, dilution ratio meter 212 indicates a 90% ratio (i.e., 90% HA and 10% PBS).

Display device 200 also displays dilution ratio increase button 214 and dilution ratio decrease button 216. In this embodiment, the user is enabled to control the specific dilution ratio by selecting dilution ratio increase button 214 and dilution ratio decrease button 216. For example, as illustrated in FIG. 2B, the user pushes dilution ratio decrease button 216. In this embodiment, modular injection system 10 displays an indication (i.e., the highlighted borders) to the user which indicates that dilution ratio decrease button 216 has been selected. In FIG. 2B, based on the selection, dilution meter 212 indicates a dilution ratio of 85% (i.e., 85% HA and 15% PBS). In this embodiment, the selection of dilution ratio decrease button 216 causes modular injection system 10 to control the extrusion of the mixed fluid such that any extruded mixed fluid has a dilution ratio of 85% (i.e., 85% HA and 15% PBS).

Display device 200 also displays injection speed setting meter 218. In this embodiment, injection speed setting meter 218 displays the current injection speed setting of the injection device. At the point in time illustrated in FIG. 2A, injection speed setting meter 218 indicates a Low speed is set for the injection device. In this embodiment, a Low speed setting corresponds to an injection rate of about 0.60 mL per minute.

Display device 200 also displays injection speed increase button 220 and injection speed decrease button 222. In this embodiment, the user is enabled to control the specific injection rate speed by selecting injection speed increase button 220 and injection speed decrease button 222. For example, as illustrated in FIG. 2C, the user pushes injection speed increase button 220. In this embodiment, modular injection system 10 displays an indication (i.e., the highlighted borders) to the user which indicates that injection speed increase button 220 has been selected. In FIG. 2C, based on the selection, injection speed setting meter 218 indicates a Medium speed is set for an injection rate. In this embodiment, a Medium speed setting corresponds to an injection rate of about 0.90 mL per minute.

In one embodiment, the modular injector system determines the ratio of the first fluid and the second fluid based on the selected injection speeds of the first fluid and the second fluid. That is, in this embodiment, the modular injection system enables a user to select a first injection rate for the first fluid and a second injection rate for the second fluid. After the injection rates have been selected or set, in response to the user selecting the inject button, the modular injection system causes each of the injectable fluids to extrude the modular injection system based on their selected injection rates.

It should be understood that, in one example, the user is enabled to cause the modular injection system to select a dilution ratio of 100% (e.g., 100% HA and 0% PBS).

In one embodiment, drive unit 140 includes a single gear motor and a transmission. For example, drive unit 1000 illustrated in FIG. 10 includes: (a) single gear motor 1002; (b) gearhead 1004; (c) output shaft 1006; (d) transmission 1008 having: (i) input configured to receive the output shaft 1006; and (ii) gear ratios; (e) first plunger 1010; and (e) second plunger 1012. In this embodiment, output shaft 1006 of single gear motor 1002 is connected to input 1006 of transmission 1008 which in turn drives second plunger 1012. In this embodiment, the transmission's gear ratios are selected such that each gear can deliver a desired dilution ratio. In another embodiment, drive unit 1000 includes a separate energy source (not shown) to switch gears in transmission 1008. In different embodiments, the gears are switched in transmission 1008 using an additional motor, a user operated switch, and/or a nitinol actuator. In other embodiments, using a single encoder (not shown) on the single gear motor 1002, the injection device determines the positions of first plunger 1010 and second plunger 1012 based on the amount of time the transmission was engaged in each gear.

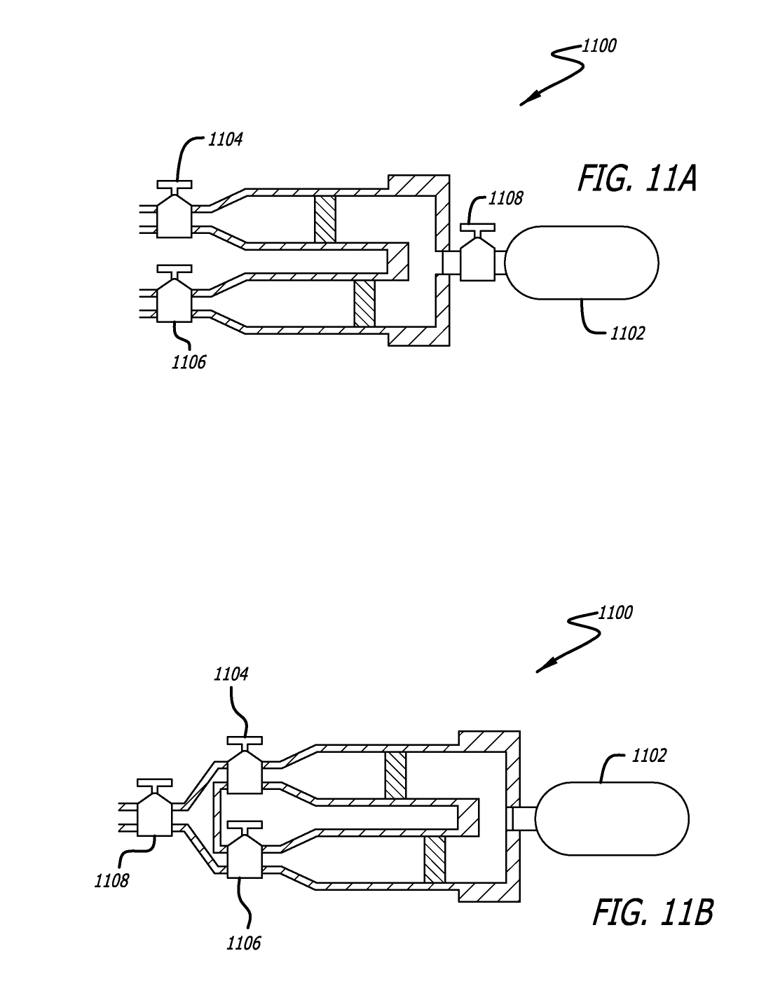

In one embodiment, drive unit 140 includes a pressure driven system which includes a pressure source (e.g., a CO2 cartridge) used to drive each plunger forward. In this embodiment, the dilution ratio is determined by regulating the flow of the fluid from each cartridge. In operation, the modular injection system enables a user to manually control the individual flow out of the cartridges using pressure/flow regulators or variable orifice valves. In one embodiment, the modular injection system electronically controls the individual flow out of the cartridges using pressure/flow regulators. Referring to FIG. 11A, in this embodiment, drive unit 1100 includes: (a) pressure source 1102; (b) first regulator 1104; (c) second regulator 1106; and (d) third regulator 1108. In operation, the net flow through modular injection system 10 is controlled by third regulator 1108 being positioned at the inlet to the cartridges. In another embodiment, as illustrated in FIG. 11B, the net flow through the modular injection system 10 is controlled by third regulator 1108 being positioned at a location after the fluids have mixed. It should be appreciated that, where the drive unit of the modular injection system includes a pressure driven system, many pressure/flow regulator combinations may be used to control injection rate and dilution ratio. In this embodiment, the modular injection system may determine the amount of fluid which has been injected/extruded using encoders which indicate the positions of the plungers.

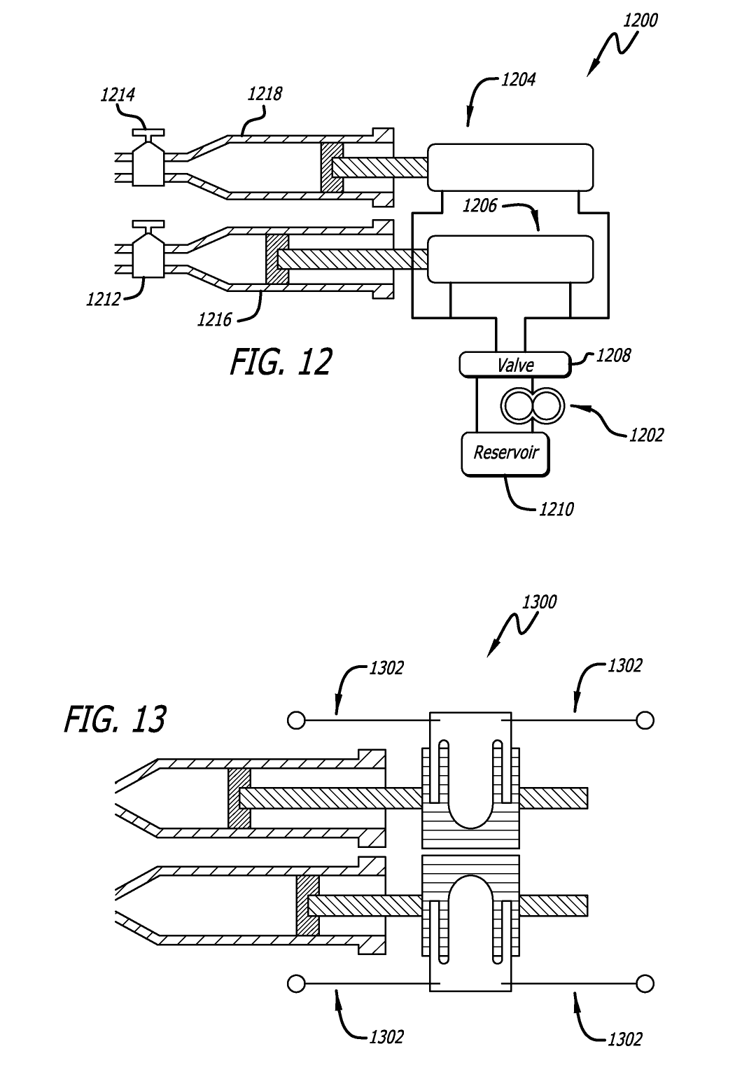

In one embodiment, drive unit 140 includes a hydraulically driven system. For example, as illustrated in FIG. 12, drive unit 1200 includes: (a) pump 1202; (b) first hydraulic piston 1204; (c) second hydraulic piston 1206; (d) valve 1208; (e) reservoir 1210; (f) first regulator 1212; and (g) second regulator 1214. In operation, modular injection system 10 uses pump 1202 to activate hydraulic pistons 1204 and 1206. The hydraulic pistons force the plungers forward which drive the fluids out of cartridges 1216 and 1218. In this embodiment, the modular injection system uses regulators 1212 and 1214 to control the flow of the fluids out of cartridges 1212 and 1214. In one embodiment, modular injection system 10 enables a user to manually control the individual flow out of the cartridges using the pressure/flow regulators. In some embodiments, the injection device electronically controls the individual flow out of the cartridges using pressure/flow regulators. In another embodiment, the dilution ratio is determined by the relative regulation of each cartridge. In other embodiments, after the completion of an injection, valve 1208 is toggled. This can allow pump 1202 to drive hydraulic fluid into the front of the pistons, retracting the plungers quickly.

In one embodiment, drive unit 140 includes a nitinol drive system. For example, as illustrated in FIG. 13, drive unit 1300 includes shape memory actuators 1302. In this embodiment, shape memory actuators 1302 are wire-shaped and are made of nitinol or some other material that changes shape or size. Although in this embodiment, four wires are illustrated, it should be appreciated that in different embodiments, the modular injection system may include any suitable amount of wire-shaped memory actuators. When an electrical current is applied to shape memory actuators 1302, the shape memory actuators 1302 shorten a specific amount. More specifically, the electric current causes shape memory actuators 1302 to heat, which in turn triggers its length transformation. Each pair of opposing wires turns off and on in sequence, causing a ratcheting member to toggle back and forth. It should be appreciated that any number of wires may be used in parallel to increase force or to increase plunger speed. In one embodiment, the injection device determines the location of the plungers by counting the number of actuations of wires 1302 and correlating the count with plunger movement.

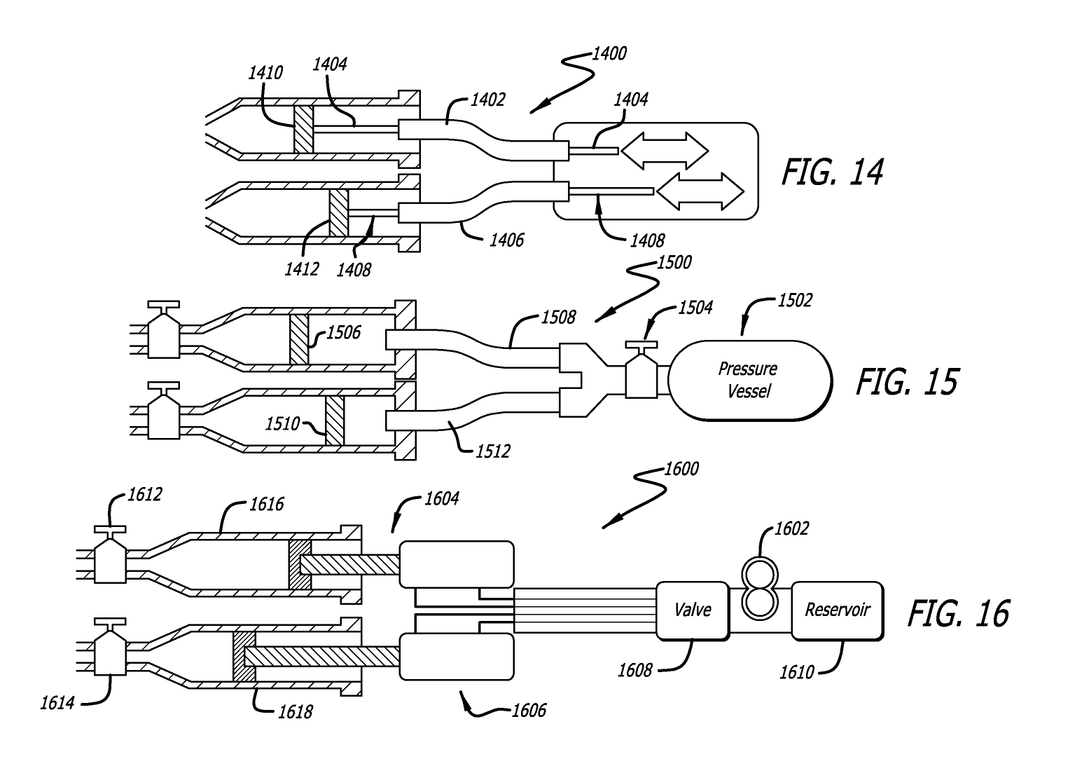

In one embodiment, the drive unit includes a wire system. For example, as illustrated in FIG. 14, drive unit 1400 includes: (a) first tube 1402 which encompasses first wire 1404; (b) second tube 1406 which encompasses second wire 1408; (c) first plunger 1410; (d) second plunger 1412; and energy source 1414. In this embodiment, control device 102 includes energy source 1414, and cartridges 1416 and 1418 are located in handheld injection device 100. In operation, energy source 1414 generates energy, and first wire 1404 transfers energy from control device 102 to handheld injection device 100 to drive first plunger 1410, thereby causing handheld injection device 100 to extrude the first injectable fluid. Second wire 1408 transfers energy from control device 102 to handheld injection device 100 to drive second plunger 1412, thereby causing handheld injection device 100 to extrude the second injectable fluid. The wires may be driven by any mechanism described above. In one embodiment, energy is transferred across a tether. In this embodiment, the sheath that comprises the tether need to be relatively stiff to transfer forces effectively.

In one embodiment, the drive unit includes a pressure vessel which transmits pressure from the control device to the handheld injection device. In one embodiment, the pressure vessel transmits pressure from the control device, through tubing, to the cartridges located in the handheld injection device. As illustrated in FIG. 15, drive unit 1500 includes: (a) pressure vessel 1502; (b) flow/pressure regulator 1504; (c) first plunger head 1506 which is driven by pressure vessel 1502 using pressure transferred using first tube 1508; and (d) second plunger head 1510 which is driven by pressure vessel 1502 using pressure transferred using second tube 1512. It should be appreciated that the method of controlling the flow out of each cartridge, and therefore the dilution ratio, may be any method described above relating to the pressure driven system.

In one embodiment, the drive unit includes a hydraulic system where fluid lines are housed within a tether between the handheld injection device and the control device. For example, as illustrated in FIG. 16, drive unit 1600 includes: (a) pump 1602; (b) first hydraulic piston 1604; (c) second hydraulic piston 1606; (d) valve 1608; (e) reservoir 1610; (f) first regulator 1612; and (g) second regulator 1614. In this embodiment, in operation, modular injection system 10 uses pump 1602 to activate hydraulic pistons 1604 and 1606. Hydraulic pistons 1604, 1606 force the plungers forward which drive the fluids out of cartridges 1616 and 1618. In this embodiment, the modular injection system uses regulators 1612 and 1614 to control the flow of the fluids out of cartridges 1616 and 1618. In one embodiment, modular injection system 10 enables a user to manually control the individual flow out of the cartridges using the pressure/flow regulators. In this embodiment, after the completion of an injection, valve 1608 can be toggled, allowing pump 1602 to drive hydraulic fluid into the front of the pistons, retracting the plungers quickly.

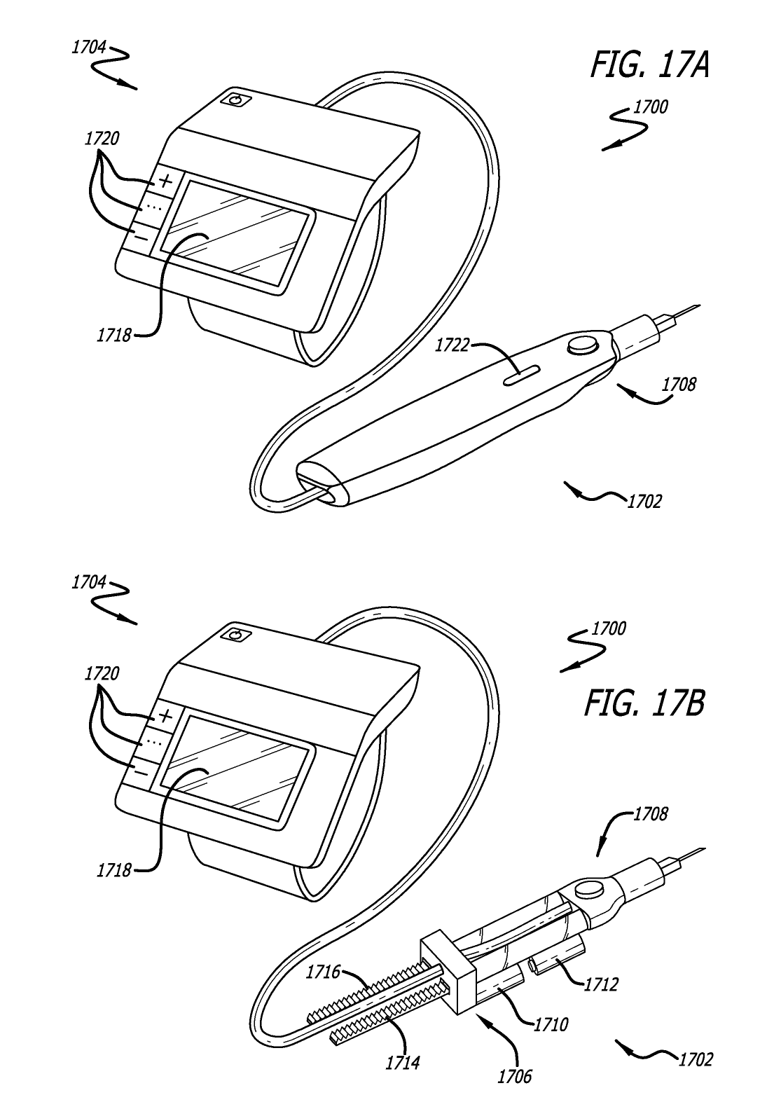

In alternative embodiments, certain components of the device are housed in alternative locations. For example, as illustrated in FIGS. 17A and 17B, modular injection system 1700 includes handheld injector device 1702 and control device 1704. In this embodiment, handheld injector device 1702 includes drive unit 1706 and mixing unit 1708. In this embodiment, drive unit 1706 is a dual gear motor system. Drive unit 1706 includes: (a) first motor 1710; (b) second motor (not shown); (c) first battery 1712; (d) second battery (not shown); (e) first rack 1714; and (f) second rack 1716.

In this embodiment, separate control device 1704 includes a portion of the control system. That is, control device 1704 includes display device 1718; input devices 1720; at least one processor (not shown); and at least one memory device (not shown). Handheld injection device 1702 can include input device or inject button 1722.

In one embodiment, where the handheld injection device includes the drive unit, components of the handheld injection device can be configured such that the weight of the handheld injection device is effectively balanced by positioning the cartridges in the front section of the handheld injection device, and the motors in the rear section of the handheld injection device. In this embodiment, the cross section of the injection device is fairly consistent along its length.

In another embodiment, the components of the handheld injection device are configured such that configuration of the handheld injection device has a front section having a diamond shaped cross section based on the positions of the motors and the cartridges. This diamond-shaped cross section may provide improved ergonomics.

In one embodiment, the input devices include at least one sensor. For example, modular injection system 10 may include a cartridge inserted sensor. Using the cartridge inserted sensor, modular injection system 10 may detect whether at least one cartridge is inserted in the cartridge housing. The cartridge inserted sensor may prevent modular injection system 10 from attempting to perform an injection without cartridge(s) properly loaded. In one embodiment, modular injection system 10 includes a home sensor. Using the home sensor, modular injection system 10 may detect whether the injection device is in a home state.

In one embodiment, modular injection system 10 includes at least one motor driver. The motor driver can communicate with both the processor and the motor(s). The motor driver may provide the systems necessary to control the operation of the motor(s). In another embodiment, using input from sensors and encoders, the processor directs the motor(s) through the motor driver, which in turn may control the extension of the plunger and thus the injection.

In addition, modular injection system 10 may include a power system. For example, modular injection system 10 may house at least one battery, or other power source (e.g., a rechargeable battery or a fuel cell). In one embodiment, the power includes electrical power. The battery may be connected to the control system in any suitable manner. For example, the battery may be permanently connected, e.g., soldered, or may be connected through a connector. In the latter case, a door may be provided in modular injection system 10, which may allow access to the battery for removal and replacement. In one embodiment, the control device may be powered by an electrical power source, and the handheld injector unit may draw electrical power from the power source using the cable.

In addition, modular injection system 10 may include a battery charger. The battery charger may be capable of charging the at least one battery when connected to an external source of electricity. For example, modular injection system 10 may include a connector, which may allow the injector device to connect to a source of electrical power, such a standard 120 or 240 V AC power source. Of course, modular injection system 10 need not connect to such a power source directly. Rather modular injection system 10 may connect to a power adaptor or supply system, which may in turn connect to the primary power source. In addition, any suitable connector may be provided, e.g., in the body of modular injection system 10, for connection to the external power source.

In one embodiment, the modular injection system includes disposable components. In one embodiment, the disposable components include anything that may come in contact with the injectable fluids (wet components). The disposable components may also include anything which is integral to the function of the wet components. For example, the disposable components may include a needle, syringes (filled with e.g., HA and PBS), plungers, o-rings, tubing, housings, fittings and/or seals.

In one embodiment, the modular injection system includes durable components which include components intended to be reused between patients. Therefore, in one embodiment, the injection device is easily cleaned. In one embodiment, the durable components include the drive unit, battery or batteries, the user interface, the printed circuit boards and any necessary electrical connections, the disposable retention mechanism for locking disposable and durable components together, and/or housings (e.g., lids, doors, slides, etc.).

The disposable components can be loaded into the durable components in any suitable way. For example, the disposable components can be loaded into the durable components employing slide in (slot) loading, drop in (shotgun) loading, and clip in loading, or any combination of these methods.

In the preceding specification, the present disclosure has been described with reference to specific example embodiments thereof. It will, however, be evident that various modifications and changes may be made thereunto without departing from the broader spirit and scope of the present disclosure. The description and drawings are accordingly to be regarded in an illustrative rather than restrictive sense.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

What is claimed is:

1. A modular injection system comprising:
a handheld injector device including a mixing unit configured to mix a first injectable fluid and a second injectable fluid; and
a control device which is separate from the handheld injector device, the control device including:
(a) a processor;
(b) an input device operatively coupled to the processor;
(c) a first cartridge defining a first chamber configured to contain the first injectable fluid;
(d) a second cartridge defining a second chamber configured to contain the second injectable fluid;
(e) a drive unit operatively coupled to the processor; and
(f) a memory device storing instructions which when executed by the processor, causes the processor, in cooperation with the input device, the first cartridge, the second cartridge and the drive unit, to:
(i) select a dilution ratio of the first injectable liquid and the second injectable liquid;
(ii) based on the selected dilution ratio, produce an injectable mixed fluid by diluting the first injectable liquid with the second injectable liquid; and
(iii) extrude the injectable mixed fluid.

2. The modular injection system of claim 1, wherein the control device is configured to be secured to a wrist of a user of the modular injection system.

3. The modular injection system of claim 2, which includes a wrist strap removably connected to the control device.

4. The modular injection system of claim 1, which includes at least one cable which connects the handheld injection device to the control device.

5. The modular injection system of claim 1, wherein the first injectable fluid includes a dermal filler.

6. The modular injection system of claim 5, wherein the dermal filler is a hyaluronic acid-based dermal filler.

7. The modular injection system of claim 1, wherein the second injectable fluid includes a phosphate buffered saline.

8. The modular injection system of claim 1, wherein the drive unit includes:
(a) a plurality of gear motors; and
(b) a plurality of racks operatively coupled to the gear motors, the plurality of racks being operatively engaged with a plurality of plungers.

9. The modular injection system of claim 1, wherein the drive unit includes a pressure source and a pressure regulator.

10. The modular injection system of claim 1, wherein the instructions, when executed by the processor, causes the processor to, in cooperation with at least one input device, select an injection rate.

11. The modular injection system of claim 10, wherein the instructions, when executed by the processor, causes the processor to extrude the injectable mixed fluid based on the selected injection rate.

12. The modular injection system of claim 1, wherein the instructions, when executed by the processor, causes the processor to, in cooperation with the at least one input device:
(a) select a first injection rate for the first injectable fluid; and
(b) select a second injection rate for the second injectable fluid.

13. A modular injection system comprising:
a handheld injector device including:
(a) a first cartridge defining a first chamber configured to contain a first injectable fluid;
(b) a second cartridge defining a second chamber configured to contain a second injectable fluid;
(c) a mixing unit configured to mix the first injectable fluid and the second injectable fluid; and
(d) a drive unit;
a control device which is separate from the handheld injector device, the control device including:
(a) a processor operatively coupled to the drive unit;
(b) an input device operatively coupled to the processor; and
(c) a memory device storing instructions which when executed by the processor, causes the processor, in cooperation with the input device, the first chamber, the second chamber and the drive unit, to:
(i) select a dilution ratio of the first injectable liquid and the second injectable liquid;
(ii) based on the selected dilution ratio, produce an injectable mixed fluid by diluting the first injectable liquid with the second injectable liquid; and
(iii) extrude the injectable mixed fluid.

14. The modular injection system of claim 13, wherein the control device is configured to be secured to a wrist of a user of the modular injection system.

15. The modular injection system of claim 14, which includes a wrist strap removably connected to the control device.

16. The modular injection system of claim 13, which includes at least one cable which connects the handheld injection device to the control device.

17. The modular injection system of claim 13, wherein the first injectable fluid includes a dermal filler.

18. The modular injection system of claim 17, wherein the dermal filler is a hyaluronic acid-based dermal filler.

19. The modular injection system of claim 13, wherein the second injectable fluid includes a phosphate buffered saline.

20. The modular injection system of claim 13, wherein the drive unit includes:
(a) a plurality of gear motors; and
(b) a plurality of racks operatively coupled to the gear motors, the plurality of racks being operatively engaged with a plurality of plungers.

21. The modular injection system of claim 13, wherein the drive unit includes a pressure source and a pressure regulator.

22. The modular injection system of claim 13, wherein the instructions, when executed by the processor, causes the processor to, in cooperation with the at least one input device, select an injection rate.

23. The modular injection system of claim 22, wherein the instructions, when executed by the processor, causes the processor to extrude the injectable mixed fluid based on the selected injection rate.

24. The modular injection system of claim 13, wherein the instructions, when executed by the processor, causes the processor to, in cooperation with the at least one input device:
(a) select a first injection rate for the first injectable fluid; and
(b) select a second injection rate for the second injectable fluid.

25. A method of operating a modular injection system including a memory device storing instructions, the method comprising:
(a) causing at least one processor to execute the instructions to select a dilution ratio of a first injectable liquid and a second injectable liquid, the at least one processor being located in a control device;
(b) causing the at least one processor to execute the instructions to, using the selected dilution ratio, produce an injectable mixed fluid by diluting the first injectable liquid with the second injectable liquid; and
(c) causing the at least one processor to execute the instructions to extrude the injectable mixed fluid, the injectable mixed fluid being extruded by a handheld injection device which is separate from the control device.
